(19) United States
(12) Patent Application Publication
(10) Pub. No.: US 2006/0122500 A1
(43) Pub. Date: Jun. 8, 2006

(54) Imaging method and apparatus for visualizing coronary heart diseases, in particular instances of myocardial infarction damage

(76) Inventors: Bjoern Heismann, Erlangen (DE); Andreas Mahnken, Aachen (DE)

Correspondence Address: HARNESS, DICKEY & PIERCE, P.L.C., P.O.BOX 8910, RESTON, VA, 20195, US

(21) Appl. No.: 11/274,203

(22) Filed: Nov. 16, 2005

(30) Foreign Application Priority Data
Nov. 17, 2004 (DE) 10 2004 055 460.9

(51) Int. Cl. A61B 5/05

(52) U.S. Cl. 600/425

(57) ABSTRACT

An imaging method and an apparatus for visualizing coronary heart diseases, in particular instances of myocardial infarction damage, are disclosed. The technique of computed tomography is used to record and reconstruct at least one image of the heart or a region of the heart, which image covers at least one part of the myocardium. Areas in the region of the myocardium that are defectively perfused and/or damaged are segmented by windowing measured data for the image or data derived therefrom, and displayed with identification in the image. It is thus possible, for example, to pictorially display the extent of the damage after a myocardial infarct.

The present application hereby claims priority under 35 U.S.C. §119 on German patent application number DE 10 2004 055 460.9 filed Nov. 17, 2004, the entire contents of which is hereby incorporated herein by reference.

### FIELD

The present invention generally relates to an imaging method for visualizing coronary heart diseases, in particular instances of myocardial infarction damage. In such a case, a technique of computed tomography may be used to record and reconstruct at least one image of the heart or of a region of the heart, which image covers at least a part of the myocardium. The invention also generally relates to an apparatus for carrying out the method.

### BACKGROUND

Imaging techniques for visualizing coronary heart diseases, in particular coronary calcification or strictures, constitute an important aid in evaluating the state of the heart. This relates both to preliminary examinations for the early recognition of circulation disturbances, and to the monitoring of a coronary heart disease, if appropriate after a bypass operation or an angioplasty, over a relatively long period. It is possible with the aid of such examinations to better estimate the risk of a heart attack, and to check the success of an operation or a therapy. Computed tomography (CT) is a known non-invasive imaging technique with which symptoms of coronary heart diseases can be visualized.

The cardiac muscle is damaged in the event of a myocardial infarction by temporary or permanent loss of perfusion. In this case, there is firstly a reduction in the perfusion, and secondly the metabolism changes. One of the consequences, an increase in the water content in the infarction area, already occurs in the acute myocardial infarction. In the further course, transformation processes take place with increased fibrosis and, finally, scar formation. However, it has not so far been possible to observe these processes directly with the aid of the technique of computed tomography. A reliable detection of the infarction area is also not a reliable possibility in contrast-enhanced computed tomography.

### SUMMARY

An object of at least one embodiment of the present invention resides in specifying a noninvasive imaging method and/or an apparatus, with the aid of which it is possible, for example, to visualize more effectively the extent of myocardial infarction damage.

An object may be achieved with the aid of a method and/or an apparatus. Advantageous refinements of the method and of the apparatus can be gathered from the following description and the example embodiments.

In the case of at least one embodiment of the present imaging method for visualizing coronary heart diseases, in particular instances of myocardial infarction damage, the technique of computed tomography may be used to record and reconstruct at least one image of the heart or a region of the heart, which image covers at least a part of the myocardium. The method may be distinguished in that areas in the region of the myocardium that are defectively perfused and/or damaged are segmented by windowing measured data for the image or data derived therefrom, and displayed with identification, in particular highlighted, in the image.

By windowing the measured data or image data or, if appropriate, data derived therefrom in the image area that covers the at least one part of the myocardium, it is possible to distinguish between healthy and damaged regions of the myocardium. In the subsequent image display, at least nonperfused and/or damaged regions of the myocardium are highlighted. In this way, the viewer can immediately recognize the effect on the myocardium of a coronary heart disease, for example the effect of vascular constrictions or vascular occlusions in the heart. In the case of a preceding myocardial infarction, it is possible in this way for the spatial extent of the damage to the myocardium to be detected at once.

A threshold value method is used for the windowing of the measured data or, if appropriate, data derived therefrom. In the case of the windowing of the measured data, at least one HU (HU: Hounsfield Unit) value region is prescribed within which the HU measured values of damaged and/or nonperfused regions of the myocardium lie in the case of a CT measurement. All the pixels or voxels of the recorded image which are based on such HU measured values are assigned to a nonperfused and/or damaged region. In addition, well perfused regions can also be segmented by prescribing corresponding HU value regions, and can be identified as healthy regions on the subsequent image display.

In at least one example embodiment of the present method, at least two congruent images of the heart or of the region of the heart are recorded, these images being based on a different spectral distribution of the x-radiation. This may be performed, for example, by image recording with different tube voltages, different x-ray tubes or different spectral characteristics of the x-ray detectors.

In one example embodiment, a so-called dual energy CT system may be used for this purpose. This system has at least two imaging systems including x-ray tube and x-ray detector with different spectral properties. A spatial distribution of the effective atomic number Z and/or the density ρ can then calculated from the spectrally different measured data of the at least two pictures.

The effective atomic number of a tissue is composed in this case of the chemical atomic numbers and atomic weights of the elements participating in the structure of the tissue. The windowing may then be performed on the basis of these data derived from the original measured data, that is to say the values of the atomic number Z and/or the density ρ. Thus, a greater difference in the effective atomic number Z between healthy and damaged regions of the myocardium is to be expected on the basis of the defective perfusion of dead areas of the myocardium.

Healthy tissue has an effective atomic number of approximately 7.7 and density ρ of 1.05 g/cm³. The atomic number of dead tissue deviates substantially downward, since the Z contribution of the blood of approximately 7.8 is lacking, and necrotic tissue generally implies a substantially lesser oxygen fraction. It is possible in this way to undertake a segmentation of the nonperfused and/or damaged tissue by a windowing of the calculated Z values in a range below 7.7. This segmentation is subsequently identified in the displayed image of the heart, which is one of the originally recorded images of the heart or heart region.

The technique of so-called ρ-Z projection, for example, can be used for calculating the spatial distribution of the effective atomic number Z and/or the density ρ, which technique can be gathered, for example, from B. J. Heismann et al., “Density and atomic number measurements with spectral x-ray attenuation method”, Journal of Applied Physics, Volume 94, Number 3, pages 2073-2079. Use is made in the ρ-Z projection of the fact that the measured data obtained with the technique of computed tomography, the attenuation coefficients μ at the location \vec{r}, are a function of the x-ray energy E radiated into the tissue and the local tissue density ρ in accordance with the following equation:
μ=μ(E,\vec{r})=(μ/ρ)(E,Z)×ρ(\vec{r}),
(μ/ρ)(E, Z) representing the energy- and material-dependent mass attenuation coefficient, and Z representing the effective atomic number.

As it is determined by its effective atomic number Z, the energy-dependent x-ray absorption of the tissue is therefore superposed by the x-ray absorption influenced by the tissue density ρ. Materials and/or tissues of different chemical and physical composition can therefore have identical attenuation values in the x-ray image. Conversely, by contrast, the material composition of an examination object cannot be inferred from the attenuation value of a single x-ray picture. However, if at least two images with a different spectral distribution of the x-radiation are taken, the two fractions ρ and Z can be separated. The result is then a spatial distribution of the effective atomic number Z, and a spatial distribution of the density ρ.

As an alternative to the ρ-Z projection, it is also possible to form the quotient of the measured data of the two images recorded with a different spectral distribution, doing so in a simple pixelwise or voxelwise fashion. The quotient μ1/μ2 obtained is essentially a measure of the atomic number. The windowing for the segmentation can then be performed via the prescription of one or more value ranges for this quotient.

In both abovenamed variant refinements, CT units may be used with two imaging systems including in each case two x-ray tubes with assigned detectors, or CT units with spectrally resolving detector systems. In the case of the latter, measured data are simultaneously acquired using the spectrally resolving detector system for at least two different spectral regions from an x-ray radiation.

The present apparatus, in at least one example embodiment, includes a computed tomography unit that may include at least two different imaging systems for image recording with a different spectral distribution, and/or a spectrally resolving detector system. The evaluation unit of the present apparatus, in at least one example embodiment, may be designed in such a way that it automatically carries out the segmentation of the nonperfused or damaged regions on the basis of prescribed threshold values, and generates image data for an image display in which the segmented regions are highlighted. In at least one example embodiment, the evaluation unit also takes over the formation of the quotient of the measured data from spectrally different image recordings, doing so in a pixelwise or voxelwise fashion, or the ρ-Z projection with the subsequent segmentation and image display in each case, as has already been described in conjunction with the method.

### BRIEF DESCRIPTION OF THE DRAWINGS

The present method and the present apparatus are explained below briefly again with the aid of an example embodiment in conjunction with the drawings, in which:

FIG. 1 shows an example of a computed tomography unit with two imaging systems such as can be used in at least one embodiment of the present method, in a perspective overall illustration; and

FIG. 2 shows an example of a flowchart for carrying out at least one embodiment of the present method.

### DETAILED DESCRIPTION OF THE EXAMPLE EMBODIMENTS

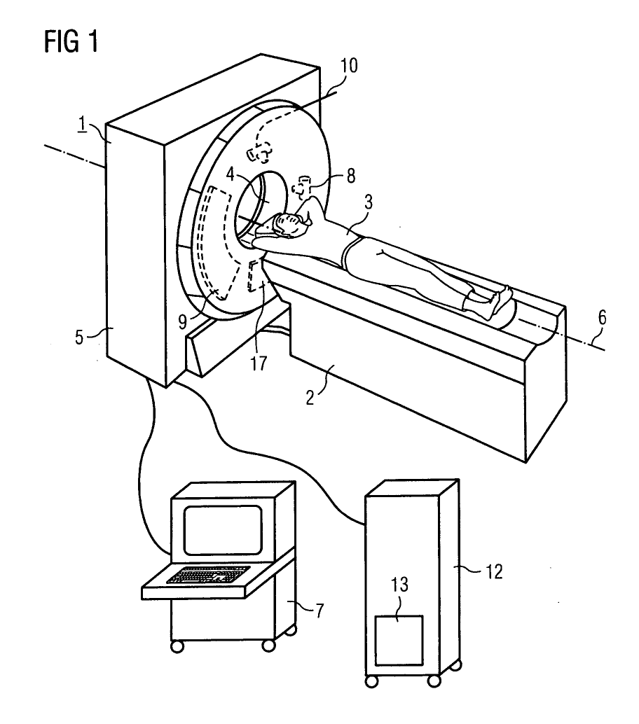

FIG. 1 shows an x-ray computed tomography unit 1 with an assigned support device 2 for accommodating and supporting a patient 3. A movable table plate of the support device 2 can be used to introduce the patient 3 with the desired examination region into an opening 4 in the housing 5 of the tomography unit 1. During a spiral scan, moreover, a continuous axial feed is undertaken with the aid of the support device 2. In the interior of the housing 5, a gantry (not visible in FIG. 1) can be rotated at high speed about a rotation axis 6 running through the patient 3. The tomography unit 1 is controlled via a control unit 7.

The tomography unit 1 has two imaging systems on the gantry that each include an x-ray tube 8 or 10 and a multirow x-ray detector 9 or 11. The arrangement of the two x-ray tubes 8, 10 and the two detectors 9, 11 on the gantry is fixed during the operation of the tomography unit 1, such that their relative spacings are also constant during operation.

In the present example, the two imaging systems are operated with a different spectral distribution, that is to say with a different x-ray voltage and/or different spectral filters in the beam path between the x-ray tube 8, 10 and the associated detector 9, 11. Of course, it is also possible to have a different spectral sensitivity of detector 9 and detector 11.

The projection data of the two continuously scanning imaging systems are further processed in a control and imaging computer 12 in accordance with at least one example embodiment of the present method and, by applying an image reconstruction algorithm, processed to form the desired image in which the damaged regions of the myocardium are identified. For this purpose, in addition to the conventional image reconstruction module the image computer 12 also includes an evaluation unit 13 designed specifically for carrying out at least one example embodiment of the present method.

In at least one example embodiment of the present method, this computed tomography unit is operated with different tube voltages and/or different spectral filter characteristics of the filters such that two images are obtained in conjunction with a different spectral distribution with each measurement scan.

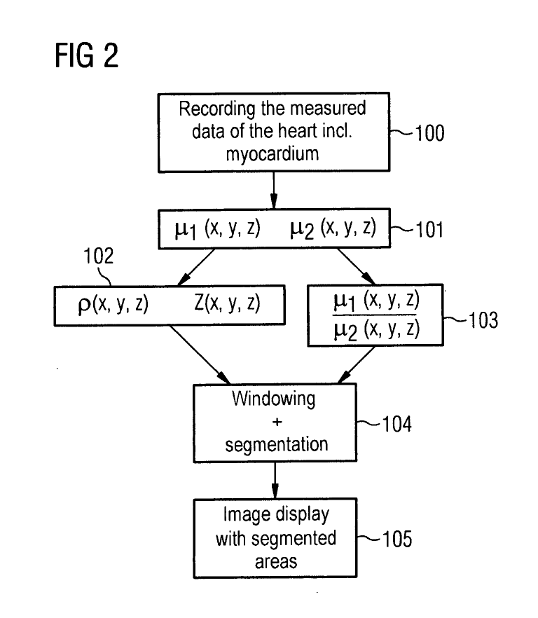

FIG. 2 shows an example of the sequence of one example embodiment of the present method in the case of which the x-ray pictures are recorded in a first step 100 with the aid of the computed tomography unit 1 illustrated in FIG. 1. Generated in this process are two images of the heart that in each case cover the myocardium and are based on a different spectral distribution of the x-radiation. Subsequently, via an image reconstruction based on the raw data obtained in relation to each of the images, in step 101 an attenuation distribution μ1 (x, y, z) or μ2 (x, y, z) of the attenuation coefficient μ is generated within the recorded 3D image or a 2D transverse tomogram with the coordinates x, y, z or x, y. In step 102, a computer-aided transformation is undertaken of the distributions of the attenuation coefficients into an atomic number distribution Z (x, y, z) and a density distribution ρ (x, y, z).

As an alternative to step 102, it is possible in step 103 to undertake a simple formation of the quotient of the attenuation data from the two images, this being done, of course, in a pixelwise or voxelwise fashion. Such a quotient formation is likewise a measure of the atomic number Z.

The following step is a windowing of the data obtained, in particular the spatial distribution of the atomic number Z (x, y, z) or the quotient μ1 (x, y, z)/μ2 (x, y, z) on the basis of prescribed threshold values within which the measured values of damaged or nonperfused myocardial tissue lie during a CT measurement. The damaged regions are segmented (step 104) on the basis of this windowing. Finally, at least one of the originally recorded images is displayed in step 105, the segmented regions being highlighted, for example in color, in the image display.

Thus, the extent of the damaged areas can be recognized at once in the displayed image. The image display can be performed both as a 2D tomogram and as a 3D volumetric image.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. An imaging method for visualizing coronary heart diseases, wherein computed tomography is used to record and reconstruct at least one image of at least one of the heart and a region of the heart, the at least one image covering at least one part of the myocardium, the method comprising:
segmenting areas in the region of the myocardium, that are at least one of defectively perfused and damaged, by windowing measured data for at least one of the image and data derived therefrom; and
displaying the areas with identification in the image.

2. The method as claimed in claim 1, wherein at least two images of at least one of the heart and the region of the heart are recorded and reconstructed with the aid of a different spectral distribution of the x-radiation, wherein at least one of a spatial distribution of an effective atomic number Z and a density pρ in the region of the myocardium is calculated from the measured data for the two images, and wherein the windowing is carried out with values of at least one of the effective atomic number Z and the density ρ.

3. The method as claimed in claim 1, wherein at least two images of at least one of the heart and the region of the heart are recorded and reconstructed with aid of a different spectral distribution of the x-radiation, wherein a quotient is formed from the measured data for the two images in the region of the myocardium in at least one of a pixelwise and voxelwise fashion, and wherein the windowing is carried out with values of the quotient.

4. The method as claimed in claim 1, wherein the at least one image is recorded with the aid of a computer tomograph that has a number of imaging systems composed of x-ray source and x-ray detector for the purpose of simultaneous image recording.

5. An apparatus for carrying out the method as claimed in claim 1, comprising
a computed tomography unit including an evaluation unit, the evaluation unit being designed to automatically carry out a segmentation of at least one of defectively perfused and damaged areas of the myocardium in a recorded image of the heart on the basis of prescribed threshold values, and generate image data for an image display in which the segmented regions are identified.

6. The apparatus as claimed in claim 5, wherein the computed tomography unit includes at least two different imaging systems for image recording with a different spectral distribution of the x-radiation.

7. The apparatus as claimed in claim 5, wherein the computed tomography unit includes a spectrally resolving detector system for image recording with a different spectral distribution of the x-radiation.

8. The apparatus as claimed in claim 5, wherein the evaluation unit is designed to form quotients from measured data of two images of the heart recorded with a different spectral distribution of the x-radiation in at least one of a pixelwise and voxelwise fashion, and wherein the segmentation is carried out on the basis of the values of the quotients.

9. The apparatus as claimed in claim 5, wherein the evaluation unit is designed to calculate a spatial distribution of at least one of an effective atomic number Z and a density ρ in the region of the myocardium from measured data of two images of the heart recorded with a different spectral distribution of the x-radiation, and carry out the segmentation on the basis of the values of at least one of the effective atomic number Z and the density ρ.

10. The method as claimed in claim 1, wherein the method is for visualizing instances of myocardial infarction damage.

11. The method as claimed in claim 2, wherein the at least one image is recorded with the aid of a computer tomograph that has a number of imaging systems composed of x-ray source and x-ray detector for the purpose of simultaneous image recording.

12. The method as claimed in claim 3, wherein the at least one image is recorded with the aid of a computer tomograph that has a number of imaging systems composed of x-ray source and x-ray detector for the purpose of simultaneous image recording.

13. The apparatus as claimed in claim 6, wherein the computed tomography unit includes a spectrally resolving detector system for image recording with a different spectral distribution of the x-radiation.

14. An imaging method for visualizing coronary heart diseases, comprising:
using computed tomography to record and reconstruct at least one image of at least one of the heart and a region of the heart, the image covering at least one part of the myocardium;
segmenting areas in the region of the myocardium, that are at least one of defectively perfused and damaged, by windowing measured data for at least one of the image and data derived therefrom, for an image display in which the segmented regions are identified.

15. The method as claimed in claim 14, wherein at least two images of at least one of the heart and the region of the heart are recorded and reconstructed with the aid of a different spectral distribution of the x-radiation, wherein at least one of a spatial distribution of an effective atomic number Z and a density ρ in the region of the myocardium is calculated from the measured data for the two images, and wherein the windowing is carried out with values of at least one of the effective atomic number Z and the density ρ.

16. The method as claimed in claim 14, wherein at least two images of at least one of the heart and the region of the heart are recorded and reconstructed with aid of a different spectral distribution of the x-radiation, wherein a quotient is formed from the measured data for the two images in the region of the myocardium in at least one of a pixelwise and voxelwise fashion, and wherein the windowing is carried out with values of the quotient.

17. The method as claimed in claim 14, wherein the at least one image is recorded with the aid of a computer tomograph that has a number of imaging systems composed of x-ray source and x-ray detector for the purpose of simultaneous image recording.

18. An imaging apparatus for visualizing coronary heart diseases, comprising:
a computed tomography unit to record and reconstruct at least one image of at least one of the heart and a region of the heart, the image covering at least one part of the myocardium, the computer tomography unit including an evaluation unit designed to automatically carry out a segmentation of at least one of defectively perfused and damaged areas of the myocardium in a recorded image of the heart on the basis of prescribed threshold values, and generate image data for an image display in which the segmented regions are identified.

19. The apparatus as claimed in claim 18, wherein the computed tomography unit includes at least two different imaging systems for image recording with a different spectral distribution of the x-radiation.

20. The apparatus as claimed in claim 18, wherein the computed tomography unit includes a spectrally resolving detector system for image recording with a different spectral distribution of the x-radiation.

21. The apparatus as claimed in claim 18, wherein the evaluation unit is designed to form quotients from measured data of two images of the heart recorded with a different spectral distribution of the x-radiation in at least one of a pixelwise and voxelwise fashion, and wherein the segmentation is carried out on the basis of the values of the quotients.

22. A computed tomography unit to record and reconstruct at least one image of at least one of the heart and a region of the heart, the image covering at least one part of the myocardium, the computer tomography unit comprising:
an evaluation unit designed to automatically carry out a segmentation of at least one of defectively perfused and damaged areas of the myocardium in a recorded image of the heart on the basis of prescribed threshold values, and generate image data for an image display in which the segmented regions are identified.

23. The computed tomography unit as claimed in claim 22, further comprising at least two different imaging systems for image recording with a different spectral distribution of the x-radiation.

24. The computed tomography unit as claimed in claim 22, further comprising a spectrally resolving detector system for image recording with a different spectral distribution of the x-radiation.

25. The computed tomography unit as claimed in claim 22, herein the evaluation unit is designed to form quotients from measured data of two images of the heart recorded with a different spectral distribution of the x-radiation in at least one of a pixelwise and voxelwise fashion, and wherein the segmentation is carried out on the basis of the values of the quotients.
